(19) United States
(12) Patent Application Publication
(10) Pub. No.: US 2026/0335558 A1
(43) Pub. Date: Oct. 29, 2026

(54) ROTATING PLUNGER LOCK

(71) Applicant: Terumo Corporation, Tokyo (JP)

(72) Inventor: James Shimabukuro, Aliso Viejo, CA (US)

(21) Appl. No.: 19/106,134

(22) PCT Filed: Aug. 24, 2023
(86) PCT No.: PCT/US2023/072812

§ 371 Date: Feb. 24, 2025

Related U.S. Application Data
(60) Provisional application No. 63/400,955, filed on Aug. 25, 2022.

(51) Int. Cl. A61M 5/315; A61M 1/00; A61M 5/31

(52) U.S. Cl. CPC A61M 5/31505; A61M 1/81; A61M 5/3137; A61M 5/31511; A61M 2005/3139

(57) ABSTRACT

A rotating plunger lock is provided with a base including a first through-hole and a socket; and a rotational lock including a third through-hole, smaller than the first through-hole, the rotational lock being fitted to the socket and secured by the cap, wherein the rotational lock is configured to rotate in the socket between a first state in which the rotational lock obstructs a portion of the first through-hole and a second state in which the rotational lock is clear of the first through-hole.

### CROSS-REFERENCES TO RELATED APPLICATIONS

The present disclosure claims the benefit of U.S. Provisional Patent Application No.: 63/400,955 entitled “ROTATING PLUNGER LOCK” and filed on 2022 Aug. 25 and of International Patent Application No. PCT/US2023/072812 entitled “ROTATING PLUNGER LOCK” and filed on 2023 Aug. 24, each of which is incorporated herein by reference in its entirety

### BACKGROUND

Negative pressure syringes can be used to provide a vacuum (e.g., for aspirating body fluids or emboli), and the plunger is drawn upward outward and held in place to generate and hold a vacuum for the procedure. Manually drawing the plunger may be difficult for some users, depending on the negative pressure to be applied, as can maintaining that pressure over the course of the procedure.

### SUMMARY

The present disclosure is generally related to an improved rotating plunger lock for use for use with a syringe and plunger system as part of a suction or aspiration device. The system includes a rotational lock so that a user can secure the plunger at a known position in the syringe to maintain vacuum more easily and precisely. When the plunger is pulled back, a rib on the plunger is passed around a locking tooth, and when the plunger reaches a maximum position, the locking tooth rotates to hold the plunger in place. Additionally, in some embodiments, the motion supplied by the user is only translation (e.g., pulling back on the plunger) and the described mechanism automatically rotates and to lock the plunger in place, thereby further easing use of the described syringe and plunger system. The improvements described herein provide various benefits, including, but not limited to: easier use of the associated devices, more precise control of suction generated by manual aspiration system, and improved or simplified manufacturing techniques.

One embodiment of the present disclosure is a device, comprising: a syringe body; a plunger, including a shaft having a first end that forms a seal with an inner surface of the syringe body, a second end opposite to the first end, and a plurality of ribs included between the first end and the second end; a rotational lock through which the shaft runs, the rotational lock including a tooth that selectively interfaces with the plunger via the plurality of ribs; and a base through which the shaft runs, the base permitting rotation of the rotational lock between a first state and a second state, wherein the first state positions the tooth to contact a given rib of the plurality of ribs and prevent translation of the plunger relative to the syringe body, and wherein the second state positions the tooth out of contact with the plurality of ribs to allow translation of the plunger relative to the syringe body.

One embodiment of the present disclosure is a device, comprising: a base including a first through-hole and a socket; and a rotational lock including a third through-hole, smaller than the first through-hole, wherein the rotational lock is fitted to the socket and is configured to rotate in the socket between a first state in which the rotational lock obstructs a portion of the first through-hole and a second state in which the rotational lock is clear of the first through-hole.

One embodiment of the present disclosure is a syringe, comprising: a syringe body; a plunger to sealingly engage with a lumen of the syringe body and slide along a longitudinal axis of the syringe body; and a selective locking means to selectively engage the plunger in a first position that restricts the plunger from sliding along the longitudinal axis, and to selectively disengage the plunger in a second position that permits the plunger to slide along the longitudinal axis.

One embodiment of the present disclosure is a syringe, comprising: a syringe body having a lumen with a longitudinal axis; a plunger at least partially within the syringe body and sealingly engaged with the lumen of the syringe body, wherein the plunger has a plurality of ribs that project outwardly from the plunger; and a selective locking mechanism having at least one tooth, where in a locked configuration the at least one tooth is engaged with at least one of the plurality of ribs such that the plunger resists sliding along the longitudinal axis, and where in an unlocked configuration the at least one tooth is disengaged from the plurality of ribs such that the plunger is slidable along the longitudinal axis.

### BRIEF DESCRIPTION OF THE DRAWINGS

The accompanying figures depict various elements of the one or more embodiments of the present disclosure, and are not considered limiting of the scope of the present disclosure.

In the Figures, some elements may be shown not to scale with other elements so as to more clearly show the details. Additionally, like reference numbers are used, where possible, to indicate like elements throughout the several Figures.

It is contemplated that elements and features of one embodiment may be beneficially incorporated in the other embodiments without further recitation or illustration. For example, as the Figures may show alternative views and time periods, various elements shown in a first Figure may be omitted from the illustration shown in a second Figure without disclaiming the inclusion of those elements in the embodiments illustrated or discussed in relation to the second Figure.

FIGS. 1A-1D provide exploded views of several configurations of an aspiration system, according to embodiments of the present disclosure.

FIGS. 2A-2C provide views of an aspiration system at various draw states, according to embodiments of the present disclosure.

FIGS. 3A and 3B illustrate a plunger lock using a compression spring-based biasing mechanism, according to embodiments of the present disclosure.

FIGS. 4A and 4B illustrate a plunger lock using two compression spring-based biasing mechanisms, according to embodiments of the present disclosure.

FIGS. 5A and 5B illustrate a plunger lock using a flexion spring-based biasing mechanism, according to embodiments of the present disclosure

FIGS. 6A and 6B illustrate a plunger lock using a magnet-based biasing mechanism, according to embodiments of the present disclosure

FIGS. 7A and 7B illustrate operation of a travel-stop on the rotational lock, according to embodiments of the present disclosure.

FIGS. 8A-8C illustrate operation of a tooth of the rotational lock with ribs of the plunger, according to embodiments of the present disclosure.

### DETAILED DESCRIPTION

The present disclosure is generally related to an improved rotating plunger lock for use for use with a syringe and plunger system as part of a suction or aspiration device. The improvements described herein provide various benefits, including, but not limited to: easier use of the associated devices, more precise control of suction generated by manual aspiration system, and improved or simplified manufacturing techniques.

The described system includes a rotational lock to secure the plunger at a known position in the syringe to maintain vacuum more easily and precisely. Ribs on the plunger can pass around a locking tooth on the rotational lock when drawn outward, and prevent the plunger from traveling inward to the syringe by the resulting negative pressure until a user manually releases the lock.

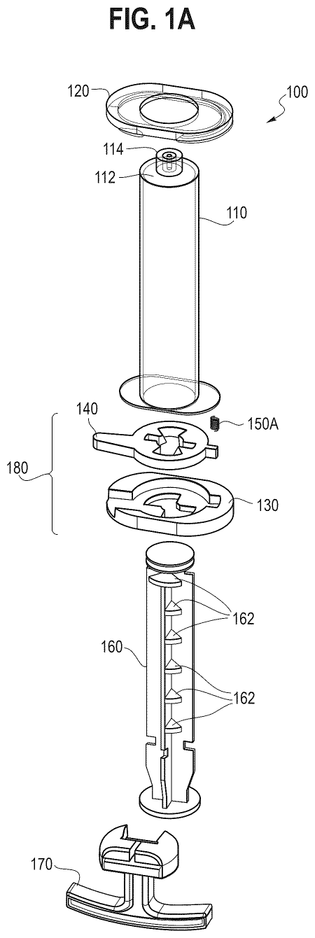

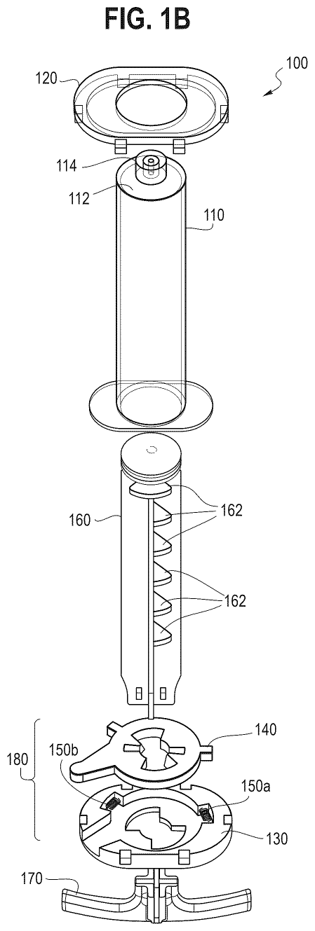

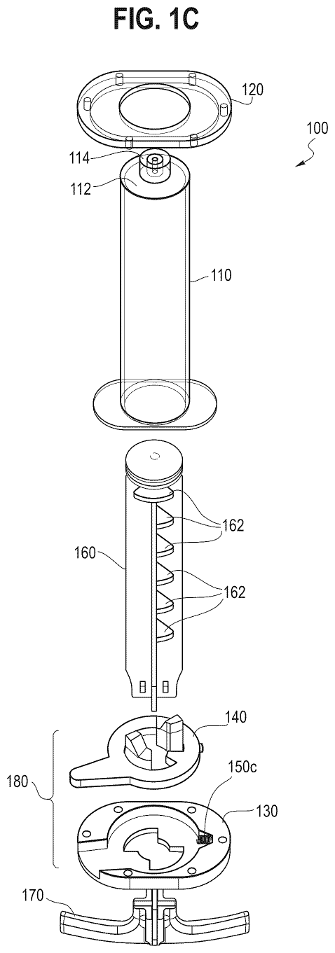

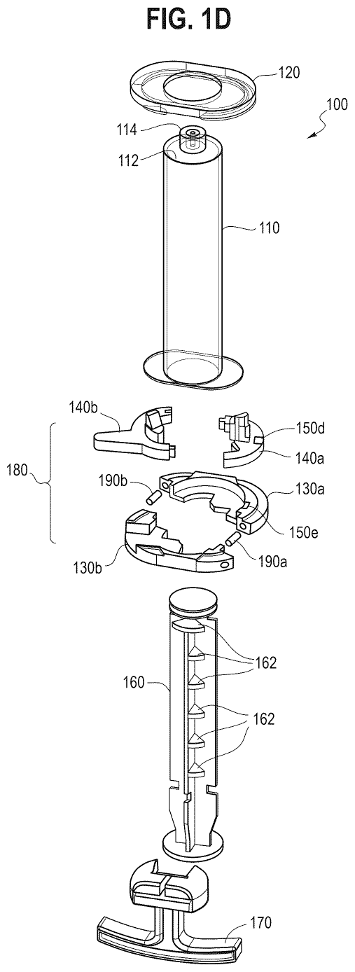

FIGS. 1A-1D provided exploded views of several configurations of an aspiration system 100, according to embodiments of the present disclosure. Each of the aspiration systems 100 in FIGS. 1A-1D include a syringe body 110, a plunger 160, a rotational lock 140 with a corresponding base 130, and a handle 170.

When assembled, the aspiration system 100 allows a user to generate a negative pressure (e.g., suction) on a fluid target (e.g., a blood vessel) to pull fluid and any carried or blocking solids (e.g., blood clots) out from the fluid target. The negative pressure is generated by forming a partial vacuum or low pressure region in a syringe body 110 by pulling an inserted plunger 160 outward while maintaining a seal between the plunger 160 and an internal wall of the syringe body 110. As used herein, “outward travel” and similar terms refer to a direction of travel for an inserted plunger 160 away from a terminus 112 of the syringe body 110, while “inward travel” and similar terms refer to a direction of travel for an inserted plunger 160 towards a terminus 112 of the syringe body 110.

Depending on the volume of the syringe body 110 and the amount of outward travel imparted by a user on the plunger 160, different levels of negative pressure may be imparted on the fluid target. For example, the aspiration system 100 may impart pressures of ±20 or ±30 pounds per square inch (psi). The pressures are communicated to the fluid target via a port 114 defined in the terminus 112 of the syringe body 110, and may be communicated via various intermediate tubes, catheters, flow control devices (e.g., stopcocks), ports, and the like to the fluid target. To aid a user in applying a desired amount of pressure to the fluid target, the syringe body 110 may include various gradations on an otherwise transparent or translucent lumen so that the location of the plunger 160 (and amount of draw on the plunger 160) can be monitored. Additionally, to aid the user in maintaining a negative pressure once applied, the base 130, rotational lock 140, and one or mode biasing mechanisms 150a-e (generally or collectively biasing mechanism 150) may interact with the plunger 160 to hold the plunger 160 in place during operation. These components (the base 130, rotational lock 140, and biasing mechanisms 150) may be referred to as a plunger lock 180 and provide a selective locking means for the assembled syringe.

In various embodiments, the plunger lock 180 is secured to the syringe body 110 via a cap 120, which connects to the base 130 around a portion of the syringe body 110 (e.g., the finger guards) via various fasteners, adhesives, or integrated connectors. In some embodiments, the plunger lock 180 is secured directly to the syringe body 110 without a cap 120 via various fasteners, adhesives, or integrated snap connectors (on the syringe body 110 and/or the base 130). The cap 120 includes a through-hole that is substantially circular in cross-sectional area with a diameter set to accommodate the syringe body 110 being inserted therethrough, while the base 130 and the rotational lock 140 have through-holes sized and shaped to permit the plunger 160 to run therethrough (at least in the unlocked configuration).

Each of the aspiration systems 100 in FIGS. 1A-1D include various biasing mechanisms 150a-d that interact with the base 130 and the rotational lock 140 to bias the rotational lock 140 into a first state that prevents or resists the plunger 160 being drawn further into, out of, or both into and out of the syringe body 110 or a second state that freely permits the plunger 160 being drawn further into and out of the syringe body 110. The plunger 160 is slidable inward or outward from the syringe body 110 when the plunger lock 180 is in an unlocked configuration, and the plunger lock 180 prevents the plunger 160 from sliding inward when in the locked configuration. The various biasing mechanisms 150 interface with elements of the base 130 and the rotational lock 140 to provide a biasing means to shift the plunger lock 180 back to a preferred or biased-for configuration when an external force shifts the plunger lock 180 to the non-preferred or biased against configuration.

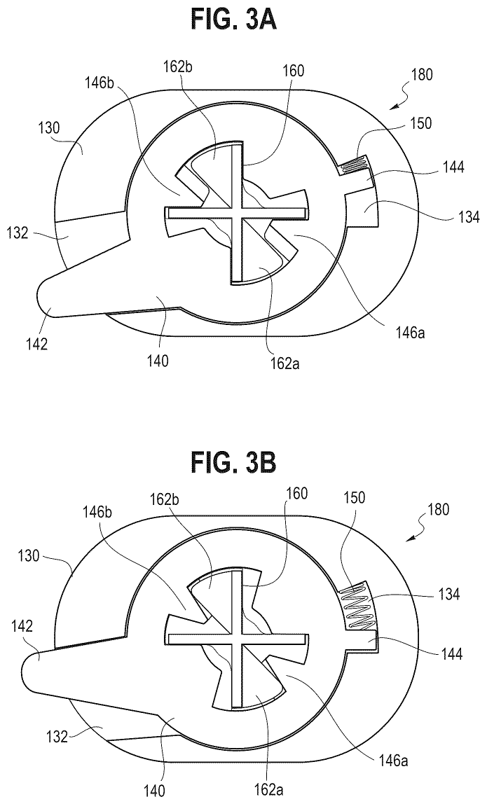

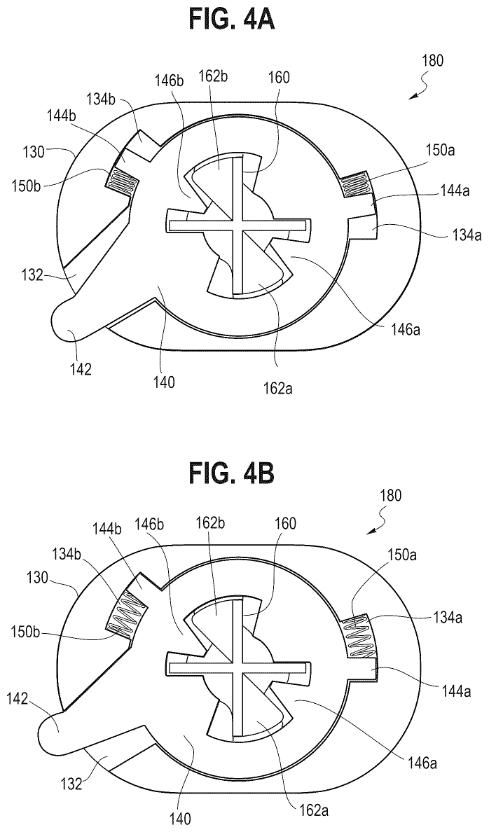

FIG. 1A illustrates a first biasing mechanism 150a of a compression spring, while FIG. 1B illustrates a first biasing mechanism 150a and a second biasing mechanism 150b that are both compression springs. Compression springs can bias the rotational lock 140 so that when a user manually rotates the rotational lock 140 into one state, the spring compress so that when the user releases pressure from the rotational lock 140, the spring expands to push the rotational lock back to the biased state. Compression springs are discussed in greater detail in regard to FIGS. 3A, 3B, 4A, and 4B.

FIG. 1C illustrates a third biasing mechanism 150c of a flexion spring, which may operate in one of three manners. In the first manner, the flexion spring is couched in the base 130 and pushes against a protrusion on the rotational lock 140 to bias the rotational lock 140 to a first one of the locked or unlocked configurations, and resists rotation to a neutral or intermediate configuration until rotated past the neutral or intermediate configuration, after which the flexion spring pushes against a protrusion on the rotational lock 140 in a second direction to bias the rotational lock 140 to a second one of the locked or unlocked configurations (and against the neutral or intermediate configuration). In the second manner, the flexion spring resists movement of the rotational lock 140 from the first state to the second state and from the second state to the first state, and deflects to permit the rotational lock 140 to reorient to the other side of the flexion spring when sufficient force is applied to move the rotational lock 140 from an initial state to a subsequent state, and returns to a neutral position to resist return to the first state until sufficient force is again applied. In the third manner, the flexion spring does not allow the rotational lock 140 to pass to the other side of the flexion spring, and resists movement from an initial biased state to the unbiased state, and returns the rotational lock 140 to the biased state after reaching the unbiased state. In various embodiments, the flexion spring can include a coil spring, a leaf spring, or a sheet of metal.

FIG. 1D illustrates a fourth biasing mechanism 150d and a fifth biasing mechanism 150e of a pair of magnets. The first magnet and the second magnet are included in the rotational lock 140 and the base 130 respectively such that they face the same polarity towards each other. Accordingly, the magnets push each other to rotate the rotational lock 140 to the first or second state and out of any intermediate states.

FIG. 1D also illustrates that the rotational lock 140 and the base 130 may be constructed of multiple parts. For example, the rotational lock 140 may include a first component 140a and a second component 140b that are joined together via various fasteners, adhesives, or integrated snap connectors. Similarly, the base 130 may include a first component 130a and a second component 130b that are joined together via various fasteners (such as dowels 190a-b as shown in FIG. 1D), adhesives, or integrated snap connectors.

The plunger 160 includes a shaft having a first end that forms a seal with an inner surface of the syringe body 110, a second end opposite to the first end to which the handle 170 can be attached. In various embodiments, the handle 170 may take different form factors, and may be attached to the second end of the plunger 160 via various fasteners, adhesives, or integrated snap connectors.

The plunger 160 is able to slide along the lumen of the syringe body 110 in both an inward direction (placing more of the plunger 160 within the syringe body 110) and an outward direction (exposing more of the plunger 160 from the syringe body 110) when the plunger lock 180 is in an unlocked configuration, and is restricted from moving when the plunger lock 180 is in a locked configuration. The rotational lock 140 rotates between the first state and the second state to place the plunger lock 180 in the locked or unlocked configuration by aligning a portion of the rotational lock 140 to cover or uncover a through-hole in the base 130 that the plunger 160 slides through.

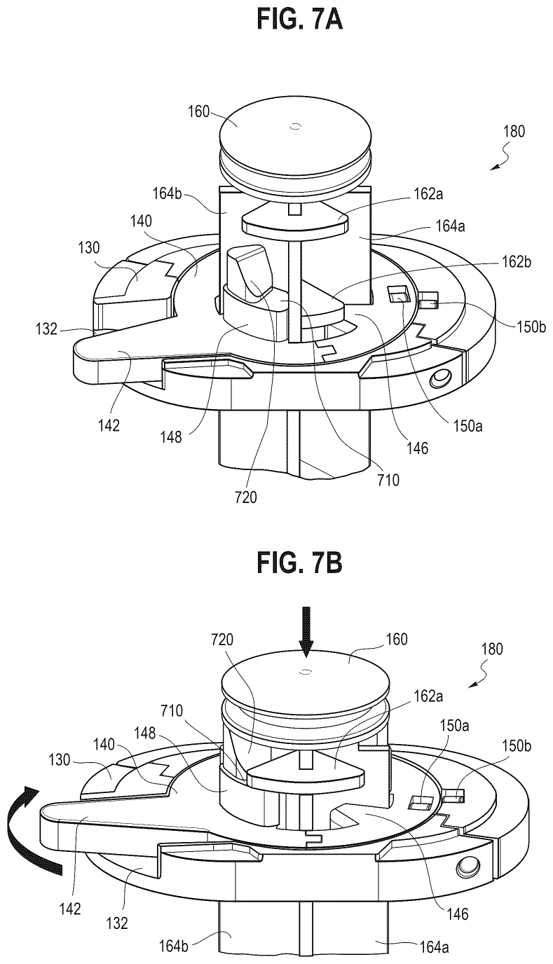

As illustrated, the plunger 160 is divided into a number of segments, and includes a plurality of ribs 162 that project outwardly from the plunger 160 in a direction substantially perpendicular to the longitudinal axis of the plunger 160. These ribs 162 interact with the rotational lock 140 when the plunger lock 180 is in the locked configuration. The ribs 162 are parallel to one another and are disposed in a first segment, which is aligned with the portion of the through-hole in the base 130 that the rotational lock 140 blocks off in the locked configuration. A lead rib 162, closest to the end of the plunger 160 that forms the seal with the syringe body 110, may optionally extend into an adjacent second segment that is aligned with a portion of the plunger lock 180 that is always blocked off, thereby keeping the plunger 160 in the syringe body 110 (e.g., mitigating overzealous operation from breaking the seal). Additionally, in some embodiments such as those as described in greater detail in regard to FIGS. 7A and 7B, the lead rib 162 interacts with a feature of the rotational lock 140 to convert the translational movement of the plunger 160 to a rotational movement in the rotational lock 140 to move the plunger lock 180 from the unlocked configuration to the locked configuration when the plunger 160 reaches its end of travel.

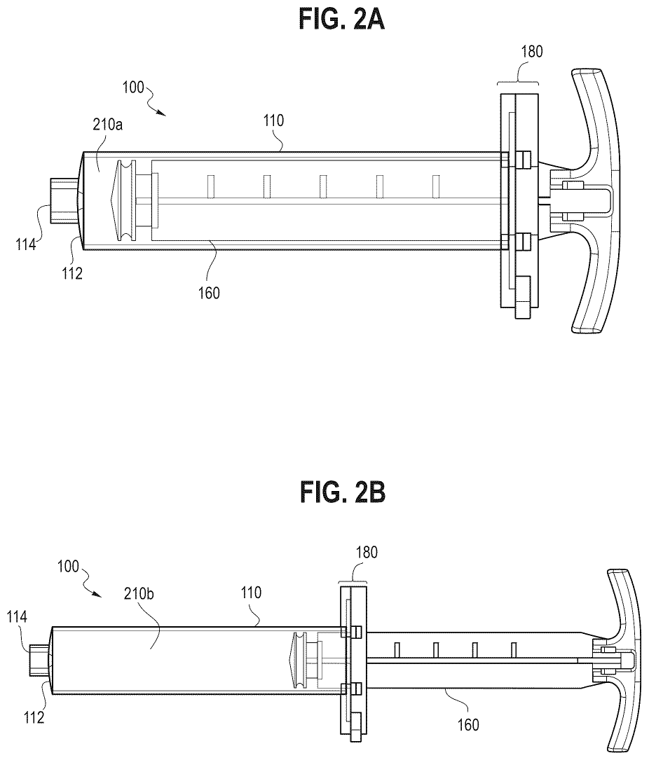

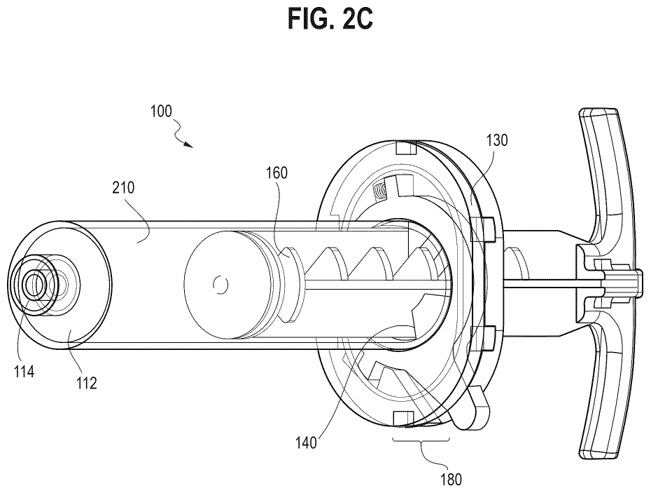

FIGS. 2A-2C provide views of an aspiration system 100 at various draws, according to embodiments of the present disclosure. FIG. 2A shows the aspiration system 100 at a zero draw state, where the plunger 160 is fully inserted into the syringe body 110, while FIG. 2B shows the aspiration system 100 at a full draw state, where the plunger 160 is fully retracted or exposed from the syringe body 110. The plunger lock 180 is affixed to the syringe body 110 and around the plunger 160 so that the plunger 160 cannot be drawn outward past the full draw state.

In various embodiments, depending on the length of the plunger 160 relative to the syringe body 110, the plunger lock 180 may prevent the plunger 160 from reaching the terminus 112 of the syringe body 110 when in the zero draw state, leaving a gap 210a (generally or collectively, gap 210). Additionally or alternatively, the user may leave a gap 210 of a predetermined size before connecting the aspiration system 100 to a fluid target to affect the pressure level that the user can apply to the fluid target at the full draw state. By reducing the potential change in the gap 210 from an initial state to a final state, the user can reduce the absolute change in pressure applied between the initial and final states. In various other embodiments, the gap 210/210a may have a predetermined size of zero or near-zero, effectively eliminating the gap 210/210a.

In addition to or alternatively to setting the size of the initial gap 210, the user may draw the plunger outward to less than the full draw state to affect the applied pressure via the difference between the initial gap 210 and final gap 210. For example, as shown in FIG. 2C, the user may draw the plunger 160 outward to an intermediate draw state between the zero draw state, where the plunger 160 could be pushed further inward or drawn further outward.

When using a manual aspiration system, the negative pressure applied to the fluid target pulls on the plunger 160; attempting to drawn the plunger 160 inward to alleviated the negative pressure. Therefore, the plunger lock 180 is provided to hold the plunger 160 in place once drawn to a desired drawn distance.

As described further in regard to FIGS. 3A-3B, 4A-4B, 5A-5B, and 6A-6B, various biasing mechanisms 150 can be employed to automatically move the rotational lock 140 to place the plunger lock 180 in the locked configuration without requiring the user to manually set the configuration. Additionally or alternatively, the chosen biasing mechanisms 150 can be employed to automatically move the rotational lock 140 to place the plunger lock 180 in the unlocked configuration without requiring the user to manually set the configuration.

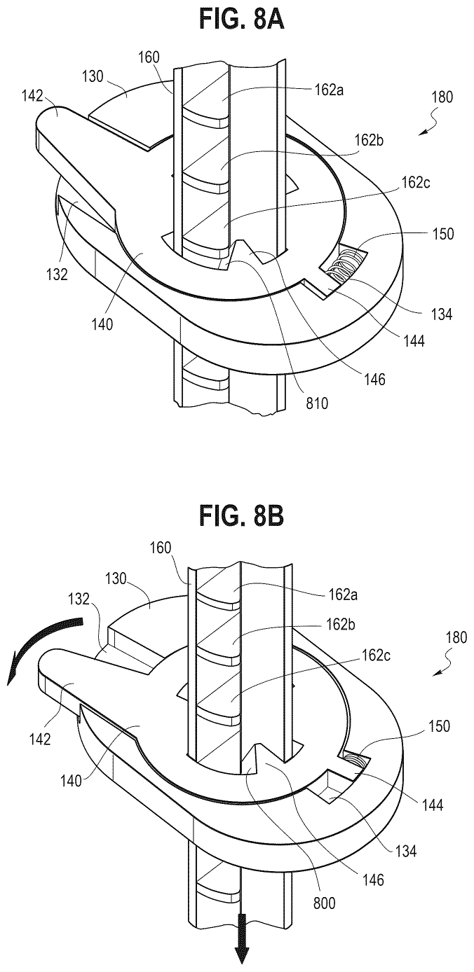

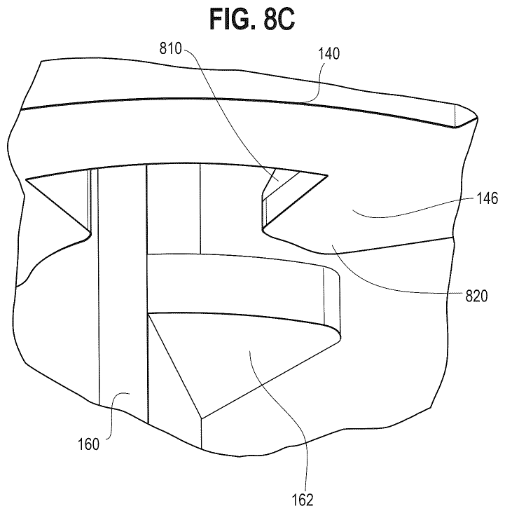

As described further in regard to FIGS. 7A-7B and 8A-8C, various configuration shifting mechanisms can be employed to automatically move the rotational lock 140 to shift the plunger lock 180 from the locked configuration to the unlocked configuration as the user draws the plunger 160 outward. When combined with biasing mechanisms 150 that return the plunger lock 180 to the locked configuration, these features allow the user of the aspiration system 100 to focus on pulling the plunger 160 to the desired draw distance without needing to manually set the locking configuration for the aspiration system 100, thereby improving ergonomics, simplifying operation, and allowing the user to assert greater force in drawing the plunger 160 outward compared to designs that require the user to manually set the locking configuration.

FIGS. 3A and 3B illustrate a plunger lock 180 using a compression spring-based biasing mechanism 150, according to embodiments of the present disclosure. FIG. 3A shows the plunger lock 180 in the unlocked configuration, while FIG. 3B shows the plunger lock 180 in the locked configuration. The base 130 includes a cavity 132 that allows for the insertion of the rotational lock 140, and for the rotation of the rotational lock 140 between the locked and unlocked configurations. In some embodiments, a handle 142 of the rotational lock 140 extends outside of the cavity 132 to allow a user to manually control whether the plunger lock 180 is in the locked or unlocked configuration by rotating the rotational lock 140.

In FIGS. 3A and 3B the base 130 includes a cavity 134 in which the biasing mechanism 150 is disposed and a bias arm 144 of the rotational lock 140 projects. When using a biasing mechanism 150 of a compression spring, the biasing mechanism 150 is placed on one side of the bias arm 144 to counter rotate the rotational lock 140 in the opposite direction. For example, in FIG. 3A, the compression spring is shown in a compressed state when the plunger lock 180 is in the unlocked configuration, and the compression spring pushes against the bias arm 144 to rotate the rotational lock 140 clockwise to return the plunger lock 180 to the locked configuration. In another example, the compression spring is located on an opposite side relative to the bias arm 144 as that shown in FIG. 3A so that the compression springs pushes against the bias arm 144 to rotate the rotational lock 140 counterclockwise to return the plunger lock 180 to the unlocked configuration when placed in the locked configuration.

Although illustrated with a compression spring as the biasing mechanism 150, the design of the rotational lock 140 and base 130 shown in FIGS. 3A and 3B may also use magnets (with facing equal or opposing polarities) in the bias arm 144 and an end face of the cavity 134 to push/pull the rotational lock 140 via electromagnetic forces to bias for the locked or unlocked configuration. Additionally or alternatively, the design of the rotational lock 140 and base 130 shown in FIGS. 3A and 3B may also use a tension spring connected to the bias arm 144 and an end face of the cavity 134 in place of the compression spring to pull the rotational lock 140 to bias for the locked or unlocked configuration rather than pushing the rotational lock 140.

The plunger 160 is shown in FIGS. 3A and 3B with a first rib 162a (generally or collectively, ribs 162) in a lower right segment, and a second rib 162b in an upper left segment (not adjacent to the lower right segment), although in other embodiments, the plunger 160 may include ribs 162 in more or fewer segments. For example, at least one lead rib 162 may occupy two adjacent segments to stop the plunger 160 from being pulled through the base 130. In another example, the plunger 160 may be divided into more or fewer segments, where the ribs 162 are disposed in one or more segments corresponding to openings in the base 130.

The rotational lock 140 includes one or more teeth 146a-b (generally or collectively, tooth 146 or teeth 146) that rotate into or out of a pathway for the ribs 162 to travel when drawing the plunger 160 outward or pushing the plunger 160 inward relative to the syringe body 110. In the locked configuration, shown in FIG. 3B, the first tooth 146a is in contact with the first rib 162a and the second tooth 146b is in contact with the second rib 162b to prevent the plunger 160 from being drawn inward to a syringe body 110 when exerting a negative pressure. In contrast, in the unlocked configuration, shown in FIG. 3A, the teeth 146 are out of contact with the respective ribs 162, thereby allow for free movement of the plunger 160 inward to the syringe body 110.

FIGS. 4A and 4B illustrate a plunger lock 180 using two compression spring-based biasing mechanisms 150a-b, according to embodiments of the present disclosure. FIG. 4A shows the plunger lock 180 in the unlocked configuration, while FIG. 4B shows the plunger lock 180 in the locked configuration. The base 130 includes a cavity 132 that allows for the insertion of the rotational lock 140, and for the rotation of the rotational lock 140 between the locked and unlocked configurations. In some embodiments, a handle 142 of the rotational lock 140 extends outside of the cavity 132 to allow a user to manually control whether the plunger lock 180 is in the locked or unlocked configuration by rotating the rotational lock 140.

When using a biasing mechanism 150 of a two or more compression springs, a first biasing mechanism 150a is placed on one side of a first bias arm 144a, and a second biasing mechanism 150b is placed on the same side of a second bias arm 144b to counter rotate the rotational lock 140 in the opposite direction. For example, in FIG. 4A, the compression springs are shown in a compressed state when the plunger lock 180 is in the unlocked configuration, and the compression springs push against the respective bias arms 144a-b to rotate the rotational lock 140 clockwise to return the plunger lock 180 to the locked configuration. In another example, the compression springs are located on an opposite side relative to the bias arms 144a-b as that shown in FIG. 4A so that the compression springs push against the bias arms 144a-b to rotate the rotational lock 140 counterclockwise to return the plunger lock 180 to the unlocked configuration when placed in the locked configuration. In various embodiments, two or more biasing mechanisms 150 may be incorporated at different locations in the plunger lock 180 to increase the biasing force over using a smaller number of similarly designed biasing mechanisms 150. Although illustrated with two compression springs, different embodiments of the plunger lock 180 may include multiple flexion springs, tension springs, magnets, or combinations of compression springs, tension springs, flexion springs, and magnets.

Similarly to FIGS. 3A and 3B, the plunger 160 in FIGS. 4A and 4B is shown with a first rib 162a in a lower right segment, and a second rib 162b in an upper left segment (not adjacent to the lower right segment), although in other embodiments, the plunger 160 may include ribs 162 is more or fewer segments. For example, at least one lead rib 162 may occupy two adjacent segments to stop the plunger 160 from being pulled through the base 130. In another example, the plunger 160 may be divided into more or fewer segments, where the ribs 162 are disposed in one or more segments corresponding to openings in the base 130.

The rotational lock 140 includes one or more teeth 146a-b that rotate into or out of a pathway for the ribs 162 to travel when drawing the plunger 160 outward or pushing the plunger 160 inward relative to the syringe body 110. In the locked configuration, shown in FIG. 4B, the first tooth 146a is in contact with the first rib 162a and the second tooth 146b is in contact with the second rib 162b to prevent the plunger 160 from being drawn inward to a syringe body 110 when exerting a negative pressure. In contrast, in the unlocked configuration, shown in FIG. 4A, the teeth 146 are out of contact with the respective ribs 162, thereby allow for free movement of the plunger 160 inward to the syringe body 110.

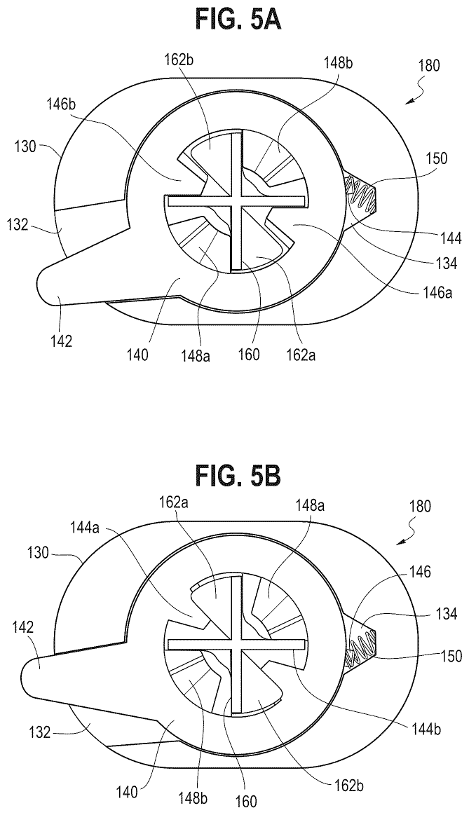

FIGS. 5A and 5B illustrate a plunger lock 180 using a flexion spring-based biasing mechanism 150, according to embodiments of the present disclosure. FIG. 5A shows the plunger lock 180 in the unlocked configuration, while FIG. 5B shows the plunger lock 180 in the locked configuration. The base 130 includes a cavity 132 that allows for the insertion of the rotational lock 140, and for the rotation of the rotational lock 140 between the locked and unlocked configurations. In some embodiments, a handle 142 of the rotational lock 140 extends outside of the cavity 132 to allow a user to manually control whether the plunger lock 180 is in the locked or unlocked configuration by rotating the rotational lock 140.

In FIGS. 5A and 5B the base 130 includes a cavity 134 in which the biasing mechanism 150 is disposed and a bias arm 144 of the rotational lock 140 projects. When using a biasing mechanism 150 of a flexion spring, the biasing mechanism 150 is placed centrally in the cavity 134 and the bias arm 144 is placed in contact with the flexion spring to bias the rotational lock 140 to that side of the cavity 134. For example, the orientation shown in FIG. 5A shows the bias arm 144 located clockwise relative to the biasing mechanism 150, which biases the plunger lock 180 to the unlocked configuration by resisting clockwise rotation. In contrast, the orientation shown in FIG. 5B shows the bias arm 144 located counterclockwise relative to the biasing mechanism 150, which biases the plunger lock 180 to the locked configuration by resisting counterclockwise rotation.

In various embodiments, the flexion spring operates in a fixed-biasing manner, where the bias arm 144 remains on one side relative to the flexion spring after being positioned. In other embodiments, the flexion spring operates in a variable-biasing manner, where the flexion spring resists rotation of the bias arm 144 for a threshold amount of rotation (e.g., +x degrees), but allows the bias arm 144 to pass to the other side of the flexion spring or for the flexion spring to reorient the direction of force applied to the bias arm 144 once the user rotates the bias arm 144 more than the threshold amount of rotation. For example, when rotating the rotational lock 140, the bias arm 144 deflects the flexion spring, which allows the bias arm 144 to pass to the other side of the flexion spring once sufficient deflection is imparted, at which time the flexion spring returns to the neutral position. In another example, when rotating the rotational lock 140, the bias arm 144 compresses the flexion spring, which allows the bias arm 144 to pass to the other side of the flexion spring once sufficient deflection is imparted, at which time the flexion spring reorients to resist return to the neutral position and the initial bias state. After passing to the other side, the flexion spring then resists rotation of the bias arm 144 for a threshold amount of rotation in the other direction (e.g., −x degrees), but allows the bias arm 144 to slip to the original side of the flexion spring once the user rotates the bias arm more than the threshold amount of rotation, thereby resetting the direction of bias.

In some embodiments, the flexion spring in a compression spring couched in the base 130 and in contact with the rotational lock 140 to push the rotational lock 140 toward a fully locked or fully unlocked configuration, and is able to reorient relative to the rotational lock 140 to push the rotational lock 140 toward the other one of the fully locked or fully unlocked configuration once a user has rotated the rotational lock 140 beyond a central or neutral orientation between the fully locked or fully unlocked configurations. In various embodiments, the flexion spring is a compression spring oriented radially to the rotational axis of the rotational lock 140, and that presses into the base 130 and the rotational lock 140 to maintain position. In some embodiments, the flexion spring is a leaf spring or a strip of metal that deflects and pushed back when the bias arm 144 is pushed into the flexion spring.

Similarly to FIGS. 3A and 3B, the plunger 160 in FIGS. 5A and 5B is shown with a first rib 162a in a lower right segment, and a second rib 162b in an upper left segment (not adjacent to the lower right segment), although in other embodiments, the plunger 160 may include ribs 162 is more or fewer segments. For example, at least one lead rib 162 may occupy two adjacent segments to stop the plunger 160 from being pulled through the base 130. In another example, the plunger 160 may be divided into more or fewer segments, where the ribs 162 are disposed in one or more segments corresponding to openings in the base 130.

The rotational lock 140 includes one or more teeth 146a-b that rotate into or out of a pathway for the ribs 162 to travel when drawing the plunger 160 outward or pushing the plunger 160 inward relative to the syringe body 110. In the locked configuration, shown in FIG. 5B, the first tooth 146a is in contact with the first rib 162a and the second tooth 146b is in contact with the second rib 162b to prevent the plunger 160 from being drawn inward to a syringe body 110 when exerting a negative pressure. In contrast, in the unlocked configuration, shown in FIG. 5A, the teeth 146 are out of contact with the respective ribs 162, thereby allow for free movement of the plunger 160 inward to the syringe body 110.

FIGS. 5A and 5B further illustrate that the rotational lock 140 may include one or more travel-stops 148a-b (generally or collectively, travel-stop 148), which are described in greater detail in regard to FIGS. 7A-7B. The travel-stops 148 are positioned to interact with lead ribs 162 that extend across more than one segment of the plunger 160 to use the translation of the plunger 160 outward from the syringe body 110 to rotate the rotational lock 140 into the locked configuration.

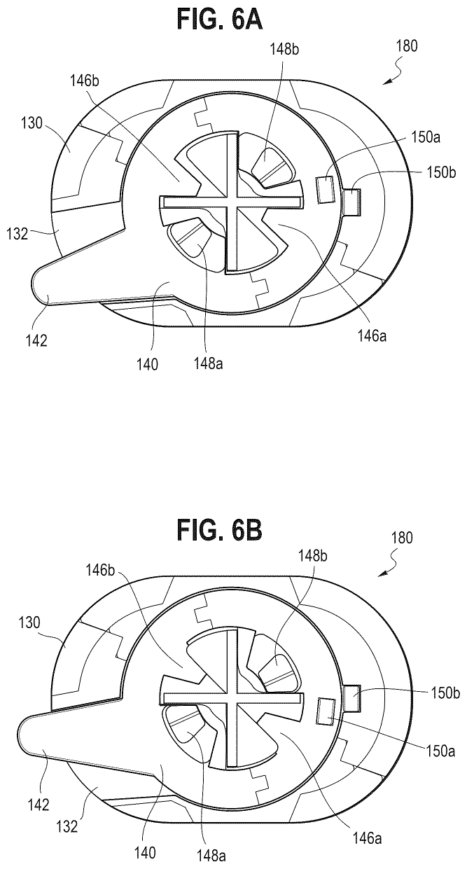

FIGS. 6A and 6B illustrate a plunger lock 180 using a magnet-based biasing mechanism 150a-b, according to embodiments of the present disclosure. FIG. 6A shows the plunger lock 180 in the unlocked configuration, while FIG. 6B shows the plunger lock 180 in the locked configuration. The base 130 includes a cavity 132 that allows for the insertion of the rotational lock 140, and for the rotation of the rotational lock 140 between the locked and unlocked configurations. In some embodiments, a handle 142 of the rotational lock 140 extends outside of the cavity 132 to allow a user to manually control whether the plunger lock 180 is in the locked or unlocked configuration by rotating the rotational lock 140.

When using a biasing mechanism 150a-b of a paired magnets (e.g., the biasing mechanisms 150d and 150e shown in FIG. 1D), a first magnet (150a) is placed in a cavity or otherwise incorporated in the rotational lock 140 and a second magnet (150b) is placed in a cavity or otherwise incorporated in the base 130 to interact with the first magnet (150a) to bias the plunger lock 180.

As illustrated in FIGS. 6A and 6B, the second magnet (150b) is located centrally in the rotational path of the rotational lock 140 between the locked and unlocked configuration, and may be oriented such that the first and second magnets have equivalent polarities facing each other (e.g., north to north or south to south). Accordingly, the first magnet (150a) is pushed by the second magnet (150b) to the one of the locked configuration or the unlocked configuration, and resists travel to the other configuration when initially placed by a user in one configuration, unless sufficient external force (e.g., via the handle 142) is exerted.

Additionally or alternatively to having a central magnet with an equivalent polarity facing the magnet (150a) in the rotational lock 140, the base 130 can include magnets with opposing polarities to the magnet (150a) in the rotational lock 140 disposed at the ends of the rotational path to draw the rotational lock 140 to one of the fully locked or fully unlocked configurations, rather than pushing the rotational lock 140 out of an intermediate configuration.

Similarly to FIGS. 3A and 3B, the plunger 160 in FIGS. 6A and 6B is shown with a first rib 162a in a lower right segment, and a second rib 162b in an upper left segment (not adjacent to the lower right segment), although in other embodiments, the plunger 160 may include ribs 162 is more or fewer segments. For example, at least one lead rib 162 may occupy two adjacent segments to stop the plunger 160 from being pulled through the base 130. In another example, the plunger 160 may be divided into more or fewer segments, where the ribs 162 are disposed in one or more segments corresponding to openings in the base 130.

The rotational lock 140 includes one or more teeth 146a-b that rotate into or out of a pathway for the ribs 162 to travel when drawing the plunger 160 outward or pushing the plunger 160 inward relative to the syringe body 110. In the locked configuration, shown in FIG. 6B, the first tooth 146a is in contact with the first rib 162a and the second tooth 146b is in contact with the second rib 162b to prevent the plunger 160 from being drawn inward to a syringe body 110 when exerting a negative pressure. In contrast, in the unlocked configuration, shown in FIG. 6A, the teeth 146 are out of contact with the respective ribs 162, thereby allow for free movement of the plunger 160 inward to the syringe body 110.

FIGS. 6A and 6B further illustrate that the rotational lock 140 may include one or more travel-stops 148a-b, which are described in greater detail in regard to FIGS. 7A-7B. The travel-stops 148 are positioned to interact with lead ribs 162 that extend across more than one segment of the plunger 160 to use the translation of the plunger 160 outward from the syringe body 110 to rotate the rotational lock 140 into the locked configuration.

FIGS. 7A and 7B illustrate operation of a travel-stop 148 on the rotational lock 140, according to embodiments of the present disclosure. As illustrated, the plunger 160 is shown with a first segment 164a that includes a series of ribs 162 with a lead rib 162a that is included in both the first segment 164a and a second segment 164b adjacent to the first segment 164a.

The travel-stop 148 is part of or connected to the rotational lock 140 and projects towards the sealing end of the plunger 160 in alignment with the second segment 164b. The travel-stop 148 includes a pedestal 710 that extends for a first distance from a base of the rotational lock 140 and interacts with the lead rib 162a to prevent movement of the plunger 160 past a certain draw distance (e.g., the full draw state). The travel-stop 148 also includes a ramp 720 that extends for a second distance (greater than the first distance) from the base of the rotational lock 140 and interacts with the lead rib 162a to rotate the rotational lock 140 as the plunger 160 is pulled outward. When the ramp 720 contacts the lead rib 162a, and the user continues to pull the plunger 160, the lead rib 162a pushes against the ramp 720 to rotate the rotational lock 140 from the unlocked configuration (that allows the plunger 160 to be pulled outward as is shown in FIG. 7A) to the locked configuration and into contact with the pedestal 710, as is shown in FIG. 7B.

FIGS. 8A-8C illustrate operation of a tooth 146 of the rotational lock 140 with ribs 162 of the plunger 160, according to embodiments of the present disclosure. As illustrated in FIGS. 8A and 8B, the plunger 160 is shown with a series of ribs 162a-c interacting with a tooth 146 of the rib 162 as the plunger 160 is pulled outward from a first perspective. FIG. 8C shows the underside of the tooth 146 relative to the first perspective shown in FIGS. 8A and 8B to provide additional detail on the operation of the tooth 146 with the ribs 162.

The tooth 146 is part of or connected to the rotational lock 140 and projects inward to a through-hole through which the plunger 160 is sized and shaped to travel. The ribs 162 are part of or connected to the plunger 160, and project radially from a longitudinal axis of the plunger 160 and are provided to reduce or prevent inward movement of the plunger 160 when interacting with a tooth 146. The tooth 146, however, includes a slope 810 on one side (e.g., an inward facing side) so that when the outward movement of the plunger 160 pushes a rib 162 against a tooth 146 while the plunger lock 180 is in the locked configuration (e.g., as in FIG. 8A), that outward movement imparts rotational movement to the rotational lock 140 to place the plunger lock 180 in the unlocked configuration. This rotation allows the rib 162 to push the tooth 146 out of the way to permit further travel of the plunger 160 outward from the syringe body 110. Although the slope 810 is illustrated in FIGS. 8A and 8B as being a substantially planar, the slope 810 may include curved or radial portions in various embodiments.

In various embodiments, when the plunger lock 180 is biased against the unlocked configuration, the rotation of the rotational lock 140 engages the biasing mechanism 150 to return to the locked configuration once the rib 162 passes the tooth 146. For example, in FIG. 8B, the rotation of the rotational lock 140 compresses a biasing mechanism 150 of a compression spring, which releases when the rib 162 moves past the tooth 146, thereby returning the plunger lock 180 to the locked configuration. Although illustrated in FIG. 5B with a compression spring as the biasing mechanism 150, other biasing mechanisms 150 and multiple biasing mechanisms 150 may be used in other embodiments.

To prevent the return of the plunger 160 inward to the syringe body 110 (e.g., due to exerting a negative pressure on a fluid target) while the plunger lock 180 is in the locked configuration, the tooth 146 includes a ratchet stop 820 on one side (e.g., an outward facing side), as is shown in FIG. 8C. Unlike the opposing (e.g., inward facing) side, the side that includes the ratchet stop 820 does not include a slope 810 so that when the rib 162 contacts the ratchet stop 820 of the tooth 146, the inward force is not translated into a rotational force onto the rotational lock 140. Although the stop 820 is illustrated as substantially planar (in a plane perpendicular to the axis of travel for the plunger 160) and described as not including a slope, it will be appreciated that edges of the tooth 146 around the ratchet stop 820 may include bevels or chamfers, which are not considered part of the ratchet stop 820.

Accordingly, the rotational lock 140 may include slopes 810 on one side of the teeth 146 and ratchet stops 820 on the other side so that a user can temporarily shift the plunger lock 180 to and from the locked and unlocked configurations while pulling the plunger 160 outward without manually setting the configuration via a handle 142 to thereby ratchet the draw state of the plunger 160 and limit unintended inward motion of the plunger 160 until the user desires to release the negative pressure.

The present disclosure may also be understood with reference to the following numbered clauses.

Clause 1: A device, comprising: a syringe body; a plunger, including a shaft having a first end that forms a seal with an inner surface of the syringe body, a second end opposite to the first end, and a plurality of ribs included between the first end and the second end; a rotational lock through which the shaft runs, the rotational lock including a tooth that selectively interfaces with the plunger via the plurality of ribs; and a base through which the shaft runs, the base permitting rotation of the rotational lock between a first state and a second state, wherein the first state positions the tooth to contact a given rib of the plurality of ribs and prevent translation of the plunger relative to the syringe body, and wherein the second state positions the tooth out of contact with the plurality of ribs to allow translation of the plunger relative to the syringe body.

Clause 2: The device of any of clauses 1 and 3-9, wherein the rotational lock includes a handle to rotate the rotational lock between the first state and the second state.

Clause 3: The device of any of clauses 1, 2, and 3-9, further comprising a biasing mechanism that biases the rotational lock to return to one of the first state and the second state when moved out of the first state or the second state by an external force.

Clause 4: The device of clause 3, wherein the biasing mechanism is one of: a return spring in contact with the rotational lock and the base, that compresses when the rotational lock is in one of the first state and the second state to return the rotational lock to the other of the first state and the second state when the external force is removed; a flexion spring in contact with the base and in contact with the rotational lock via a protrusion extending from an outer diameter of the rotational lock to bias the rotational lock to return to one of the first state and the second state when the external force is removed; or a first magnet included in the rotational lock aligned to face a first polarity outward from a central axis and a second magnet included in the base aligned to face the first polarity inward toward the central axis, wherein the first magnet is located at a first location when the rotational lock is in the first state and at a second location when the rotational lock is in the second state and the second magnet is located at a midpoint of a travel path between the first location and the second location to bias the rotational lock to the first state and the second state when rotated.

Clause 5: The device of clause 4, wherein the flexion spring operates according to one of: a first manner, wherein the flexion spring is couched in the based and pushes against a first side or a second side of the protrusion to bias the rotational lock to return to the first state or the second state, respectively, until an external force rotates the rotational lock past a neutral configuration; a second manner, wherein the flexion spring resists movement of the rotational lock from the first state to the second state and from the second state to the first state, and deflects to permit the rotational lock to reorient to the other side of the flexion spring when sufficient force is applied to rotate the rotational lock past the neutral position; and a third manner, wherein protrusion is located on one side of the flexion spring and the flexion spring does not allow the protrusion to pass to the other side of the flexion spring.

Clause 6: The device of any of clauses 1-5 and 7-9, wherein: the shaft of the plunger is divided into four segments; each rib of the plurality of ribs occupies a first portion of a first segment in a corresponding plane perpendicular to a direction that the plunger moves relative to the syringe body when translated; and the tooth is sized to be equal to or smaller in size to a remaining portion of the first segment that is not occupied by each rib in the corresponding plane.

Clause 7: The device of clause 6, wherein: a lead rib of the plurality of ribs that is located closest to an end of the plunger that forms a seal with the syringe body occupies a second portion of a second segment adjacent to the first segment; and the rotational lock includes a travel-stop that selectively interfaces with the lead rib in the second segment to rotate the rotational lock from the second state into the first state as the shaft is translated outward relative to the syringe body.

Clause 8: The device of any of clauses 1-7 and 9, wherein the tooth includes a bevel on one side that allows the plurality of ribs to rotate the rotational lock from the first state to the second state when translating the plunger outward relative to the syringe body.

Clause 9: The device of any of clauses 1-8, wherein the base is secured to a finger guard of the syringe and secures the rotational lock between the base and the finger guard.

Clause 10: A device, comprising: a base including a first through-hole and a socket; and a rotational lock including a third through-hole, smaller than the first through-hole, wherein the rotational lock is fitted to the socket and is configured to rotate in the socket between a first state in which the rotational lock obstructs a portion of the first through-hole and a second state in which the rotational lock is clear of the first through-hole.

Clause 11: The device any of clauses 10 and 12-16, wherein the rotational lock includes a handle to rotate the rotational lock between the first state and the second state, wherein the handle protrudes from the base.

Clause 12: The device of any of clauses 10, 11, and 13-16, further comprising: a cap including a second through-hole aligned on an axis with the first through-hole, the cap connected on one side of the base perpendicular to the axis, wherein the rotational lock is secured by the cap in the socket.

Clause 13: The device of clause 12, wherein: the second through-hole is substantially circular in cross-section and configured to a diameter of a syringe; and the first through-hole and the third through-hole are configured to a cross-section of a shaft of a plunger associated with the syringe, wherein the plunger includes a plurality of ribs on the shaft that interface with the rotational lock when in the first state and the plunger is translated on the axis.

Clause 14: The device any of clauses 10-13, 15, and 16, further comprising a return spring in contact with the rotational lock and the base that compresses when the rotational lock is in the second state to bias the rotational lock to return to the first state.

Clause 15: The device of any of clauses 10-14 and 16, further comprising a bias spring in contact with the base and in contact with the rotational lock on one side of a protrusion extending from an outer diameter of the rotational lock to bias the rotational lock to return to one of the first state and the second state when moved to the other of the first state and the second state by an external force.

Clause 16: The device of any of clauses 10-15, wherein: the rotational lock includes a first magnet aligned to face a first polarity outward from a central axis; the first magnet is located at a first location when the rotational lock is in the first state and at a second location when the rotational lock is in the second state; the base includes a second magnet aligned to face the first polarity inward towards the central axis; and the second magnet is located at a midpoint of a travel path between the first location and the second location to bias the rotational lock to the first state and the second state when rotated.

Clause 17: A syringe, comprising: a syringe body; a plunger to sealingly engage with a lumen of the syringe body and slide along a longitudinal axis of the syringe body; and a selective locking means to selectively engage the plunger in a first position that restricts the plunger from sliding along the longitudinal axis, and to selectively disengage the plunger in a second position that permits the plunger to slide along the longitudinal axis.

Clause 18: The syringe of any of clauses 17 and 19-21, further comprising a biasing means that biases the selective locking means to return to one of the first position and the second position when moved out of the first position or the second position by an external force.

Clause 19: The syringe of clause 18, wherein the biasing means is one of: a compression spring in contact with the selective locking means, that compresses when the selective locking means is in the second position to return the selective locking means to the first position when the external force is removed; a flexion spring in contact with the selective locking means via a protrusion extending from an outer diameter of the selective locking means to bias the selective locking means to return to one of the first position and the second position when the external force is removed; or a first magnet included in the selective locking means aligned to face a first polarity outward from a central axis and a second magnet included in the base of the selective locking means aligned to face the first polarity inward toward the central axis, wherein the first magnet is located at a first location when the selective locking means is in the first position and at a second location when the selective locking means is in the second position and the second magnet is located at a midpoint of a travel path between the first location and the second location to bias the selective locking means to the first position and the second position when rotated.

Clause 20: The syringe of any of clauses 17-19 and 21, wherein: a shaft of the plunger is divided into four segments and includes a rib that occupies a first portion of a first segment in a corresponding plane perpendicular to the longitudinal axis; and the selective locking means includes a tooth that engages the rib when the selective locking means is in the first position and is disengaged from the rib when in the selective locking means is in the second position.

Clause 21: The syringe of clause 20, further comprising a second rib that is located closer to where the plunger sealingly engages with the lumen of the syringe body than the rib, wherein the second rib occupies a second portion of a second segment adjacent to the first segment; and the selective locking means includes a bias arm to interface with the second rib in the second segment to rotate the selective locking means from the second position into the first position as the plunger slides outward relative to the syringe body.

Clause 22: A syringe, comprising: a syringe body having a lumen with a longitudinal axis; a plunger at least partially within the syringe body and sealingly engaged with the lumen of the syringe body, wherein the plunger has a plurality of ribs that project outwardly from the plunger; and a selective locking mechanism having at least one tooth, where in a locked configuration the at least one tooth is engaged with at least one of the plurality of ribs such that the plunger resists sliding along the longitudinal axis, and where in an unlocked configuration the at least one tooth is disengaged from the plurality of ribs such that the plunger is slidable along the longitudinal axis.

Clause 23: The syringe of clause 22, wherein the selective locking mechanism shifts from the unlocked configuration to the locked configuration when the plunger reaches a first selected draw state within the syringe body.

The descriptions and illustrations of one or more embodiments provided in this disclosure are intended to provide a thorough and complete disclosure the full scope of the subject matter to those of ordinary skill in the relevant art and are not intended to limit or restrict the scope of the subject matter as claimed in any way. The aspects, examples, and details provided in this disclosure are considered sufficient to convey possession and enable those of ordinary skill in the relevant art to practice the best mode of the claimed subject matter. Descriptions of structures, resources, operations, and acts considered well-known to those of ordinary skill in the relevant art may be brief or omitted to avoid obscuring lesser known or unique aspects of the subject matter of this disclosure. The claimed subject matter should not be construed as being limited to any embodiment, aspect, example, or detail provided in this disclosure unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently.

Having been provided with the description and illustration of the present disclosure, one of ordinary skill in the relevant art may envision variations, modifications, and alternate embodiments falling within the spirit of the broader aspects of the general inventive concept provided in this disclosure that do not depart from the broader scope of the present disclosure.

As used in the present disclosure, a phrase referring to “at least one of” a list of items refers to any set of those items, including sets with a single member, and every potential combination thereof. For example, when referencing “at least one of A, B, and C” or “at least one of A, B, or C”, the phrase is intended to cover the sets of: A, B, C, A-B, B-C, A-C, and A-B-C, where the sets may include one or multiple instances of a given member (e.g., A-A, A-A-A, A-A-B, A-A-B-B-C-C-C, etc.) and any ordering thereof.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

As used in the present disclosure, the terms “substantially”, “approximately”, “about”, and other relative terms encompass values within ±5% of a stated quantity, percentage, or range unless a different approximation is explicitly recited in relation to the state quantity, percentage, or range or if the context of the value indicates that a different approximation would be more appropriate. For example, a value identified as about X % may be understood to include values between 0.95*X % and 1.05*X % or between X−0.05X and X+0.05X percent, but may stop at zero or one hundred percent in various contexts. In another example, a feature described as being substantially parallel or perpendicular to another feature shall be understood to be within ±9 degrees of parallel or perpendicular. Any value stated in relative terms shall be understood to include the stated value and any range or subrange between the indicated or implicit extremes.

As used in the present disclosure, all numbers given in the examples (whether indicated as approximate or otherwise) inherently include values within the range of precision and rounding error for that number. For example, the number 4.5 shall be understood to include values from 4.45 to 4.54, while the number 4.50 shall be understood to include values from 4.495 to 4.504. Additionally, any number or range that explicitly or by context refers to an integer amount (e.g., approximately X users, between about Y and Z states), shall be understood to round downward or upward to the next integer value (e.g., X±1 users, Y−1 and Z+1 states).

The following claims are not intended to be limited to the embodiments shown herein, but are to be accorded the full scope consistent with the language of the claims. Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various aspects described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

We claim:

1. A device, comprising:
a syringe body;
a plunger, including a shaft having a first end that forms a seal with an inner surface of the syringe body, a second end opposite to the first end, and a plurality of ribs included between the first end and the second end;
a rotational lock through which the shaft runs, the rotational lock including a tooth that selectively interfaces with the plunger via the plurality of ribs; and
a base through which the shaft runs, the base permitting rotation of the rotational lock between a first state and a second state, wherein the first state positions the tooth to contact a given rib of the plurality of ribs and prevent translation of the plunger relative to the syringe body, and wherein the second state positions the tooth out of contact with the plurality of ribs to allow translation of the plunger relative to the syringe body.

2. The device of claim 1, wherein the rotational lock includes a handle to rotate the rotational lock between the first state and the second state.

3. The device of claim 1, further comprising a biasing mechanism that biases the rotational lock to return to one of the first state and the second state when moved out of the first state or the second state by an external force.

4. The device of claim 3, wherein the biasing mechanism is one of:
a return spring in contact with the rotational lock and the base, that compresses when the rotational lock is in a first one of the first state and the second state to return the rotational lock to a second one of the first state and the second state when the external force is removed;
a flexion spring in contact with the base and in contact with the rotational lock via a protrusion extending from an outer diameter of the rotational lock to bias the rotational lock to return to one of the first state and the second state when the external force is removed; or
a first magnet included in the rotational lock aligned to face a first polarity outward from a central axis and a second magnet included in the base aligned to face the first polarity inward toward the central axis, wherein the first magnet is located at a first location when the rotational lock is in the first state and at a second location when the rotational lock is in the second state and the second magnet is located at a midpoint of a travel path between the first location and the second location to bias the rotational lock to the first state and the second state when rotated.

5. The device of claim 1, wherein:
the shaft of the plunger is divided into four segments;
each rib of the plurality of ribs occupies a first portion of a first segment in a corresponding plane perpendicular to a direction that the plunger moves relative to the syringe body when translated; and
the tooth is sized to be equal to or smaller in size to a remaining portion of the first segment that is not occupied by each rib in the corresponding plane.

6. The device of claim 5, wherein:
a lead rib of the plurality of ribs that is located closest to an end of the plunger that forms a seal with the syringe body occupies a second portion of a second segment adjacent to the first segment; and
the rotational lock includes a travel-stop that selectively interfaces with the lead rib in the second segment to rotate the rotational lock from the second state into the first state as the shaft is translated outward relative to the syringe body.

7. The device of claim 1, wherein the tooth includes a bevel on one side that allows the plurality of ribs to rotate the rotational lock from the first state to the second state when translating the plunger outward relative to the syringe body.

8. The device of claim 1, wherein the base is secured to a finger guard of the syringe and secures the rotational lock between the base and the finger guard.

9. A device, comprising:
a base including a first through-hole and a socket; and
a rotational lock including a third through-hole, smaller than the first through-hole, wherein the rotational lock is fitted to the socket and is configured to rotate in the socket between a first state in which the rotational lock obstructs a portion of the first through-hole and a second state in which the rotational lock is clear of the first through-hole.

10. The device of claim 9, wherein the rotational lock includes a handle to rotate the rotational lock between the first state and the second state, wherein the handle protrudes from the base.

11. The device of claim 9, further comprising:
a cap including a second through-hole aligned on an axis with the first through-hole, the cap connected on one side of the base perpendicular to the axis, wherein the rotational lock is secured by the cap in the socket.

12. The device of claim 11, wherein:
the second through-hole is substantially circular in cross-section and configured to a diameter of a syringe; and
the first through-hole and the third through-hole are configured to a cross-section of a shaft of a plunger associated with the syringe, wherein the plunger includes a plurality of ribs on the shaft that interface with the rotational lock when in the first state and the plunger is translated on the axis.

13. The device of claim 9, further comprising a return spring in contact with the rotational lock and the base that compresses when the rotational lock is in the second state to bias the rotational lock to return to the first state.

14. The device of claim 9, further comprising a flexion spring in contact with the base and in contact with the rotational lock on one side of a protrusion extending from an outer diameter of the rotational lock to bias the rotational lock to return to a first one of the first state and the second state when moved to the a second one of the first state and the second state by an external force.

15. The device of claim 9, wherein:
the rotational lock includes a first magnet aligned to face a first polarity outward from a central axis;
the first magnet is located at a first location when the rotational lock is in the first state and at a second location when the rotational lock is in the second state;
the base includes a second magnet aligned to face the first polarity inward towards the central axis; and
the second magnet is located at a midpoint of a travel path between the first location and the second location to bias the rotational lock to the first state and the second state when rotated.

16. A syringe, comprising:
a syringe body;
a plunger to sealingly engage with a lumen of the syringe body and slide along a longitudinal axis of the syringe body; and
a selective locking means to selectively engage the plunger in a first position that restricts the plunger from sliding along the longitudinal axis, and to selectively disengage the plunger in a second position that permits the plunger to slide along the longitudinal axis.

17. The syringe of claim 16, further comprising a biasing means that biases the selective locking means to return to one of the first position and the second position when moved out of the first position or the second position by an external force.

18. The syringe of claim 17, wherein the biasing means is one of:
a compression spring in contact with the selective locking means, that compresses when the selective locking means is in the second position to return the selective locking means to the first position when the external force is removed;
a flexion spring in contact with the selective locking means via a protrusion extending from an outer diameter of the selective locking means to bias the selective locking means to return to one of the first position and the second position when the external force is removed; or
a first magnet included in the selective locking means aligned to face a first polarity outward from a central axis and a second magnet included in a base of the selective locking means aligned to face the first polarity inward toward the central axis, wherein the first magnet is located at a first location when the selective locking means is in the first position and at a second location when the selective locking means is in the second position and the second magnet is located at a midpoint of a travel path between the first location and the second location to bias the selective locking means to the first position and the second position when rotated.

19. The syringe of claim 16, wherein:
a shaft of the plunger is divided into four segments and includes a rib that occupies a first portion of a first segment in a corresponding plane perpendicular to the longitudinal axis; and
the selective locking means includes a tooth that engages the rib when the selective locking means is in the first position and is disengaged from the rib when in the selective locking means is in the second position.

20. The syringe of claim 19, further comprising a second rib that is located closer to where the plunger sealingly engages with the lumen of the syringe body than the rib, wherein the second rib occupies a second portion of a second segment adjacent to the first segment; and
the selective locking means includes a bias arm to interface with the second rib in the second segment to rotate the selective locking means from the second position into the first position as the plunger slides outward relative to the syringe body.
